(19) United States
(12) Patent Application Publication
(10) Pub. No.: US 2023/0349801 A1
(43) Pub. Date: Nov. 2, 2023

(54) DETERMINING RHEOLOGICAL PROPERTIES OF FLUIDS

(71) Applicant: Norce Innovation AS, Stavanger (NO)

(72) Inventor: Eric Cayeux, Sandnes (NO)

(73) Assignee: Norce Innovation AS, Stavanger (NO)

(21) Appl. No.: 17/800,721

(22) PCT Filed: Feb. 24, 2021
(86) PCT No.: PCT/NO2021/050048

§ 371 Date: Aug. 18, 2022

(30) Foreign Application Priority Data
Feb. 24, 2020 (NO) 20200215

(51) Int. Cl. G01N 9/26; G01N 1/14; G01N 1/42; G01N 1/44; G01N 33/28; E21B 21/01

(52) U.S. Cl. CPC G01N 9/26; G01N 1/14; G01N 1/42; G01N 1/44; G01N 33/2823; E21B 21/01

(57) ABSTRACT

A rheological property, for example a thixotropy property, of a fluid such as a drilling fluid, can according to various examples be determined by performing a method that includes providing measurement apparatus having a flowline having a first diameter section and a second diameter section; pumping the drilling fluid along the flowline; measuring at least one differential pressure in the drilling fluid upstream in the second diameter section, using at least one upstream sensor; measuring at least one differential pressure in the drilling fluid downstream in the second diameter section, using at least one downstream sensor; and using the differential pressures from the second diameter section to determine the rheological property and/or the thixotropy property. A method of determining a gelation property of the fluid is also described. Various apparatus for use in determining the rheological property, the thixotropy property, and gelation property are also exemplified.

### TECHNICAL FIELD

The present invention relates to the determination of rheological properties of fluids such as for example drilling fluids.

### BACKGROUND AND PRIOR ART

The properties of fluid circulating in a fluid system include for instance composition, density, rheological behaviour, which depends on flow rate, temperature, pressure, and circulation history of the fluid. The behaviour of certain fluid can also be affected by the properties and configuration of the path on along which the fluid is circulated. Relationships between properties in fluid systems can in many cases be modelled, and in this way, when one or more of the properties of the fluid or system are known, other properties of the fluid or system may be predicted. However, assumptions about the system or other limitations of models can lead to some models being poorly suited or yielding poor predictions in some circumstances.

In the oil and gas exploration and production industry, the ability to estimate the downhole pressure in borehole drilling operations as a function of the flow rate of drilling fluid, rotational speed of the drill string, or axial movement of the drill string can be important for keeping the annular pressure between geological pressure margins, i.e. the pore and collapse pressure on one side and the fracturing pressure on the other side.

Frictional pressure loss in a pipe, or in an annulus, depends on the rheological behaviour of the fluid. Drilling fluids are non-Newtonian and have typically a shear thinning with yield stress behaviour. Therefore, the fluid velocity field in a cross-section of a structure (e.g. a borehole with a drill string inserted therein) can be complex, with possibly a plugged zone in the region of highest velocity, i.e. a zone where the shear rate is zero, and zones where the fluid is stagnant, such as on the narrow side of an eccentric annulus.

For estimation of the frictional pressure losses it is typically sought to measure regularly the rheological behaviour of the drilling fluid. In the North Sea, it is common to measure the rheological behaviour of the drilling fluid four times per day with a Model 35 mechanical rheometer. In practice the rheological behaviour of the fluid may change substantially during a six-hour period. Pressure loss gradient estimates in a mud report from continuous rheological measurements indicate that the mud report may not always be representative of the general rheological behaviour of the drilling fluid pumped into the well.

Another concern is related to transient effects on frictional pressure losses. Most drilling fluids are thixotropic, i.e. their rheological behaviour depends on their shear history. It is rather common that some form of torsional and axial drill-string vibrations exists during drilling operations. For instance, during stick-slip, the drill-string may stop rotating for a few seconds for thereafter spins at several hundred revolutions per minute (RPM) for a few seconds. In small hole sizes, these abrupt changes of drill-string rotational speed can modify the whole fluid velocity field in a substantial way, and because of its thixotropic behaviour, the drilling fluid may not then respond as one might expect from the rheological behaviour measured in a rheometer in steady state conditions. Large variations of measured downhole equivalent circulating density (ECD) during a drilling operation in the North Sea has experienced substantial levels of torsional oscillations as reported by the dynamic sub in the bottom hole assembly (BHA). The inventor has found that prior methods may not adequately account for effects of drill-string vibrations effects upon frictional pressure losses.

Drilling fluids are typically subject to very large variations of pressure and temperature when they are circulated in a well. As with any other fluid, their rheological behaviour depends on pressure and temperature conditions. Most drilling operations only perform rheological measurements at atmospheric pressure and a given temperature, typically 50° C. To estimate the in situ frictional pressure losses at different conditions of pressures and temperatures, it may then be necessary to extrapolate the rheological behaviour of the drilling fluid. Yet these extrapolations may often not be so precise, simply because small variations in the drilling fluid composition may impact its rheological behaviour at different pressures and temperatures.

It is common to add relatively large solid particles to the drilling fluid, like for instance lost circulation materials (LCM), when there are losses from the wellbore to the formation, either because of naturally fractured rocks or after a loss circulation incident. Solid particles larger than 200 μm cannot be put in a narrow gap Couette rheometer as they would jam the measurements. Therefore, Model 35 rheograms are obtained from a fluid where the largest solid particles have been filtered out. This is unfortunate as the presence of solid particles affects the apparent rheological behaviour of a fluid.

The inventor has in this light identified a need for techniques to better measure determine the drilling fluid rheological properties, on a continuous basis, and the dependence of those properties on shear history, pressure and temperature, while requiring the least possible filtering of the fluid.

### SUMMARY OF THE INVENTION

According to a first aspect of the invention, there is provided a method of determining at least one rheological property, for example a thixotropy property, of a drilling fluid, the method comprising the steps of: providing measurement apparatus comprising a flowline comprising a first diameter section and a second diameter section; pumping the drilling fluid along the flowline; measuring at least one differential pressure in the drilling fluid upstream in the second diameter section, using at least one upstream sensor; measuring at least one differential pressure in the drilling fluid downstream in the second diameter section, using at least one downstream sensor; and using the differential pressures from the second diameter section to determine the rheological property and/or the thixotropy property.

Typically, the method may further comprise using the differential pressures from the second diameter section to determine the rheological property and/or the thixotropy property and/or thixotropic behaviour of the drilling fluid for increasing shear rates or decreasing shear rates, e.g. from the first diameter section to the second diameter section.

Typically, the first diameter section is a large diameter section and the second diameter section is a small diameter section. The method may further comprise using the differential pressures from the second diameter section to determine the thixotropic behaviour of the drilling fluid for increasing shear rates, e.g. increasing between the first diameter section and the second diameter section.

In that case, the flowline may include a third diameter section which may have a diameter that may be larger than the second diameter section and may be smaller than the first diameter section. Thus, the large diameter section may typically have a diameter significantly larger than the second diameter section, and the third diameter section may typically have a diameter significantly larger than the second diameter section. The third diameter section may typically be at least 50% larger than the second diameter section, and the first section may for example be at least 50% larger than the third diameter section. Adjoining diameter sections, e.g. the first and second diameter sections and/or the second and third diameter sections, may typically be connected to one another through a smooth diameter transition along the flowline with an angle of taper in the range typically up to and not exceeding 45° with respect to the centre longitudinal axis through the flowline.

The diameter transition may comprise a frustoconical section. The transition may be tapered in the direction along the flowline, e.g. reduce in diameter monotonically from the first diameter section to the second diameter section, or inverse tapered, e.g. increase in diameter monotonically from the second diameter section to the third diameter section. The transition may be provided within a distance along the flowline of less than one times the diameter of any of the first, second, or third diameter sections.

The method may then further comprise any one or more of: communicating the drilling fluid through the first diameter section, thereafter the second diameter section, thereafter the third diameter section; measuring at least one differential pressure in the drilling fluid upstream in the third diameter section using at least one upstream sensor, and measuring at least one differential pressure of the drilling fluid downstream in the third diameter section using at least one downstream sensor; and using the differential pressures from the third diameter section from the third diameter section to determine the thixotropic behaviour of the drilling fluid for decreasing shear rates, e.g. decreasing between the second diameter section and the third diameter section.

Thus, the method may further comprise using the differential pressures from the second diameter section, where the drilling fluid may have transitioned from the first diameter section to the second diameter section, to determine the rheological and/or thixotropic behaviour of the drilling fluid in conditions where the shear history may be from low to high shear rates. The method may further comprise using the differential pressures from the second diameter section and/or the differential pressures from third diameter section where the drilling fluid may have transitioned from the second diameter section to the third diameter section to determine the rheological and/or thixotropic behaviour of the drilling fluid in conditions where the shear history may be from high to low shear rates. Advantageously, through pumping the fluid through the first, second, and/or third diameters in the flowline, the fluid may be subjected to conditions of flow, e.g. at diameter transition locations between sections, that may emphasise the thixotropic behaviour of the drilling fluid or allow it to be observed, whereby differential pressure measurements obtained from either or both the upstream and downstream sensors may then be used to obtain data that may respond to or allow detection of the thixotropic behaviour.

The method may further comprise any one or more of: using at least one pump to pump the drilling fluid along the flowline; measuring at least one volumetric flow rate either upstream or downstream of the pump(s), preferably at a location near the pump(s); changing the pump rate of the pump from a first pump rate to a second pump rate; obtaining measurements of the differential pressures at the first pump rate; and after changing the pump rate, obtaining measurements of the differential pressures at the second pump rate.

The method may further comprise any one or more of: stepping up the flow rate along the flowline, using at least one pump; obtaining a temporal development in at least some of the measured differential pressures in response to the stepped-up flow rate; using the temporal development to determine the thixotropic property of the drilling fluid.

The method may further comprise any one or more of: obtaining at least one mass density measurement either upstream or downstream of the pump(s); using the mass density measurement to determine the Reynolds number along the flowline and determine whether the flow conditions of the drilling fluid are laminar, transitional, or turbulent.

The method may further comprise any one or more of: heating and/or cooling the fluid to change temperature conditions of the drilling fluid in the flowline; and performing the measurements of the differential pressures before and after changing the temperature conditions.

The method may further comprise: letting the fluid rest to produce gelation of the drilling fluid in at least one section of the flowline; and after gelation: starting the pumping of the drilling fluid; directing the drilling fluid with the gelation history into the second diameter section; and obtaining data comprising a temporal development, the data obtained from either or both the first and second pressure differential sensors; and using the data to obtain the thixotropy property of the fluid with the gelation history.

The method may advantageously be performed in a measurement environment, to determine the rheological property of the drilling fluid at one or more conditions of a work environment, e.g. downhole in a borehole being drilled. The method may then further comprise: providing a rheological model. The model preferably may have a thixotropy component. Measurements obtained from the differential pressure sensors at one or more temperatures and at one or more flow rates, may then be used to facilitate determining one or more parameters of the rheological model, in particular the thixotropy component of the model. A model fitting the data may be obtained. The model may comprise a curve or profile, e.g. shear stress versus shear rate of a rheological property for different pressure and/or temperature conditions of the drilling fluid. The pressure and temperature conditions in the work environment, e.g. wellbore, may generally be quite different, typically higher, than that of the measurement environment. Using the curve, fitted to the measurement data, the rheological property may be determined for the drilling fluid at one or more other pressure and temperature conditions, e.g. by extrapolation. The rheological model and the measurements obtained from the measurement apparatus may thus be used for determining the rheological property of the drilling fluid in conditions of the work environment.

The model may comprise a non-Newtonian model, optionally one with a yield stress, modified by an amplification or other modification factor that is dependent upon thixotropy. Thus, the modification factor may alter the non-Newtonian model at low shear rates for better determination of a model fitting the measurement data.

According to a second aspect of the invention, there is provided apparatus for determining at least one rheological property, for example a thixotropy property, of a drilling fluid, the apparatus comprising: a flowline comprising a first diameter section and a second diameter section; pressure sensors for measuring an upstream pressure differential and a downstream pressure differential in the second diameter section of the conduit; and determiner means for determining the rheological property, or the thixotropy property, of the drilling fluid based on the measured pressure differentials.

The apparatus may be measurement apparatus. The apparatus may further comprise at least one pump for pumping the drilling fluid through the flowline. The pump may be operable to have one or more different pump rates for controlling pressure conditions and/or flow of the drilling fluid through the flowline.

The apparatus may further comprise at least one heater/cooler for changing temperature conditions of the drilling fluid in the flowline.

The apparatus may further comprise at least one temperature sensor for obtaining at least one measurement of temperature of the drilling fluid in the flowline.

The apparatus may further comprise a choke operable in use to produce a back pressure in the drilling fluid in the flowline.

The apparatus may further comprise at least one element, e.g. valve, or restrictor or the like, for producing turbulence, shearing and/or reducing pressure in the drilling fluid upstream of the first and second diameter sections for starting fluid flow fluid in a defined shear history condition.

The apparatus may further comprise means for determining at least one gelation property of the drilling fluid at different bulk temperatures and pressures of the drilling fluid in the flowline.

The first diameter section may comprise a super large diameter section with a diameter greater than the second diameter section, and optionally greater than a third diameter section, for obtaining sheared fluid in the super large diameter section at a low bulk velocity.

The apparatus may further comprise means for determining the density of the fluid in the conduit. The apparatus and/or the means for determining density thereof, may comprise at least one densitometer.

According to a third aspect of the invention, there is provided a wellbore drilling system including the apparatus, e.g. measurement apparatus, in accordance with the second aspect of the invention. The wellbore drilling system may comprise a fluid circulation system and the apparatus of the second aspect of the invention may be connected to the drilling fluid circulation system for circulating drilling fluid into and out of a wellbore for drilling operations. The flowline of the apparatus may be arranged in fluid communication with the wellbore for supplying or receiving drilling fluid from the borehole into the flowline of the measurement apparatus for measurement and/or communicating the measured drilling fluid from the apparatus onward for use in the wellbore.

According to a fourth aspect of the invention, there is provided a method of determining at least one gelation property of a drilling fluid, the method comprising: providing measurement apparatus comprising a flowline; entering the drilling fluid into the flowline; letting the drilling fluid rest in the flowline, producing thereby gelation of the drilling fluid; pumping the drilling fluid through along the flowline after a period of rest and gelation; and obtaining measurements of at least one property of the drilling fluid in the flowline to determine the gelation property.

The flowline may comprise first and second diameter sections, and the method may further comprise any one or more of: measuring at least one differential pressure in the drilling fluid at both upstream and downstream locations along at least the second diameter section, using at least one sensor; and using the measurements of the differential pressures to determine the gelation property.

The method may further comprise any one or more of: starting the pumping of the drilling fluid after a period of rest and gelation of the drilling fluid; and using a time evolution of measured pressure differentials after the pumping is started, to determine the gelation property.

The method may further comprise stopping the pumping to place the drilling fluid at rest in the apparatus for producing gelation.

The method may further comprise, repeatedly: stopping pumping to place the drilling fluid at rest in the apparatus for producing gelation; starting the pumping of the drilling period after a period of rest of the drilling fluid; measuring a time evolution of the pressure differentials after the pumping is started, to determine the gelation property. The repeated steps may further include measuring the duration of rest. The method may further comprise measuring the time evolution of the pressure differentials for different durations of rest.

The method may further comprise obtaining the measurements at different conditions of pressure and temperature in the apparatus.

The method may further include operating at least one pump to control either or both of: pressure conditions; and flow and rest periods of the drilling fluid.

The method may further comprise producing turbulence prior to entering a super large diameter section by which the sheared fluid may be subject to very low bulk velocity, for thereafter entering the second diameter section and then passing through into a third diameter section.

According to a fifth aspect of the invention, there is provided a method of determining at least one rheological property, for example a thixotropy property, of a fluid, the method comprising the steps of: providing measurement apparatus comprising a flowline comprising a first diameter section and a second diameter section; pumping the fluid along the flowline; measuring at least one differential pressure in the fluid upstream in the second diameter section, using at least one upstream sensor; measuring at least one differential pressure in the fluid downstream in the second diameter section, using at least one downstream sensor; and using the differential pressures from the second diameter section to determine the rheological property and/or the thixotropy property.

According to a sixth aspect of the invention, there is provided apparatus for determining at least one rheological property, for example a thixotropy property, of a fluid, the apparatus comprising: a flowline comprising a first diameter section and a second diameter section; pressure sensors for measuring an upstream pressure differential and a downstream pressure differential in the second diameter section of the conduit; and determiner means for determining the rheological property, or the thixotropy property, of the fluid based on the measured pressure differentials.

According to an seventh aspect of the invention, there is provided a method of determining at least one gelation property of a fluid, the method comprising: providing measurement apparatus comprising a flowline; entering the fluid into the flowline; letting the fluid rest in the flowline, producing thereby gelation of the fluid; pumping the fluid through along the flowline after a period of rest and gelation; and obtaining measurements of at least one property of the fluid in the flowline to determine the gelation property. The flowline may comprise first and second diameter sections, and the method may further comprise any one or more of: measuring at least one differential pressure in the fluid at both upstream and downstream locations along at least the second diameter section, using at least one sensor; and using the measurements of the differential pressures to determine the gelation property.

The fluid of any of the fifth to seventh aspects may be a drilling fluid. The fluid may be another fluid as specified anywhere herein. The fluid may be a slurry, e.g. a cement slurry. The fluid may be a wellbore fluid.

According to an eighth aspect of the invention, there is provided apparatus for performing the method in accordance with first, fourth, fifth or seventh aspects of the invention.

According to a ninth aspect of the invention, there is provided a computer program for use in performing the method of the first, fourth, fifth or seventh aspects of the invention for determining the rheological property or the thixotropy property of the fluid, e.g. by using, processing and/or analysing obtained data from the sensor(s).

According to a tenth aspect of the invention, there is provided a computer device operable to execute the computer program of the ninth aspect of the invention for determining the rheological property or the thixotropy property of the fluid, e.g. by using, processing and/or analysing obtained data from the sensor(s).

According to an eleventh aspect of the invention, there is provided a rheological model obtained from or for use in performing the method of the first, fourth, fifth or seventh aspects of the invention. The rheological model may be calibrated using data from the method. The rheological model may be fitted to obtained data from the method.

According to a twelfth aspect of the invention, there is provided a data carrier or medium carrying the rheological model of the eleventh aspect. The rheological model may comprise a flow curve or a rheogram.

Any of the aspects of the invention may include any one or more further features as described in relation to any other aspect of the invention, wherever described herein.

Embodiments of the invention are advantageous in various ways as will be appreciated from throughout the specification.

### DRAWINGS AND DESCRIPTION

There will now be described, by way of example only, embodiments of the invention with reference to the accompanying drawings, in which:

FIG. 1 is a schematic representation of measurement apparatus for determining a rheological, in particular a thixotropy property of a drilling fluid;

FIG. 2 is a schematic representation of another measurement apparatus for determining a rheological property, e.g. thixotropy property, of a drilling fluid;

FIG. 3 is a schematic representation of the measurement apparatus of FIG. 2 in smaller scale and with additional detail;

FIG. 4 is a flow diagram of an operational method for operating the measurement apparatus of FIG. 3; and

FIG. 5 is a close-up representation of a diameter transition between first and second diameter sections in the measurement apparatus.

### MEASUREMENT APPARATUS AND OPERATIONAL USE

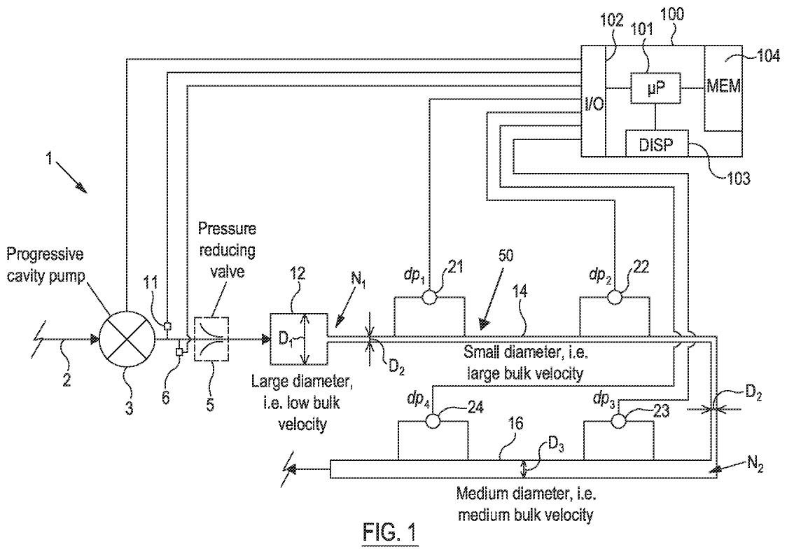

Referring first to FIG. 1, measurement apparatus 1 is provided to determine rheological properties of a drilling fluid which is supplied via a supply pipe 2 from a well (not shown). The drilling fluid is typically thixotropic, and the apparatus 1 is arranged to facilitate the detection and determination of the thixotropic behaviour of the drilling fluid, that is, the rheological properties of the fluid that are time and/or shear history dependent, and/or gelation history dependent. Indeed, it may in practice not be apparent whether the drilling fluid is thixotropic at all, and thus, the apparatus 1 can be utilised to determine that the drilling fluid exhibits thixotropy and the thixotropy properties of that thixotropic fluid, such as the degree of time dependency of rheological properties, etc. under various conditions.

The measurement apparatus 1 is in the form of a pipe rheometer or flowline rheometer which comprises a flowline 50 comprising sections of pipe with different diameters and comprising an internal passageway for the drilling fluid to travel through the apparatus 1.

The flowline 50 is formed so that that the drilling fluid enters a large diameter section 12, then enters and flows along a small diameter section 14, then enters and flows along a medium diameter section 16. In this way, the drilling fluid is exposed to different conditions of flow and stress.

The cross-section and diameter are uniform along each of the respective sections of pipe. That is, the diameter of a given section of pipe does not change in any appreciable or significant way. The purpose being to provide suitable measurement conditions for measuring properties of the fluid in the various sections. The small and medium diameter sections 14, 16, over the lengths in which measurements of the drilling fluid are to be made, are straight.

The large diameter section 12 has a super large diameter D1 that is larger than that (D2) of the small diameter section 14, typically by a factor of more than four, preferably up to 10 or more, and larger than that (D3) of the medium diameter section 16, typically by a factor of more than two, or preferably more than four. The medium diameter section 16 has a diameter D3 that is larger than that (D2) of the small diameter section, typically by a factor of more than two, or preferably more than four.

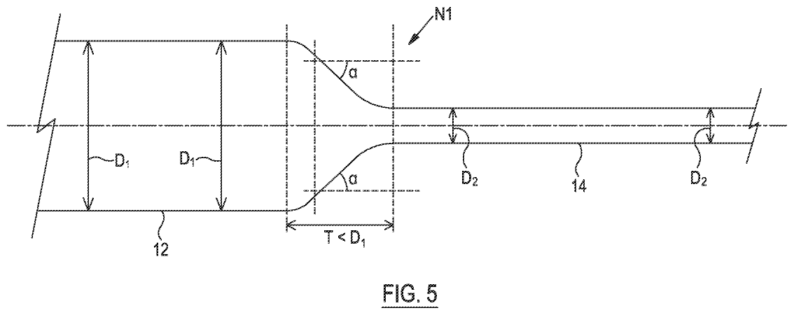

Between the respective sections of the flowline 50, there are diameter transitions N1, N2, where the diameter of the passage inside the sections decreases rapidly and/or monotonically from the large diameter section 12 to the small diameter section 14 (N1), and increases monotonically but quickly from the small diameter section 14 to the medium diameter section 16 (N2). The diameter transition N1 is exemplified in FIG. 5, where the transition from diameter D1 of the first section 12 to the diameter D2 of the second section 14 is over the distance T (less than D1) with an angle of taper a. Flow is laminar through the transitions N1, N2. This is facilitated also by the transitions being smooth.

The arrangement of the diameter transition N2 from the diameter D2 of the second section 14 to the diameter D3 of the third section 16 is similar, but has an inverse angle of taper in the downstream direction as the diameter increases.

The diameter transitions N1, N2 are fast or sudden so that the diameters change over a short distance along the pipeline from the one section to the other, which provides significant changes in flow conditions of the fluid as it travels into the next section. This allows thixotropic behaviour of the fluid to be measured in the downstream section of pipe. For instance, that taper angle is not larger than 45°, to avoid generating turbulence, but no longer than the diameter of the largest pipe, so that the transition is sufficiently quick to allow approximating the shear rate history through the tapered section as a step change. The first transition N1 is over the distance of at least less than the diameter of the small diameter section 14, and the second transition N2 is over the distance of at least less than the diameter of the small diameter section 14 or the medium diameter section 16.

Moving on then to consider the apparatus 1 further, first and second differential pressure sensors 21, 22 are provided for measuring differential pressures in the fluid in the small diameter section 14 at upstream and downstream locations along the section 14.

The apparatus also includes third and fourth differential pressure sensors 23, 24 provided for measuring differential pressures in the fluid in the medium diameter section 16 at upstream and downstream location of the section 16.

The apparatus 1 also has a pump 3 positioned upstream of the sections of pipe, i.e. upstream of the large diameter section 12. The pump 3 is arranged to pump the drilling fluid through the apparatus 1. The pump 3 receives drilling fluid from the supply pipe 2 and pumps the fluid through the various pipe sections in the apparatus 1.

The apparatus 1 also has a volumetric flowmeter 11 placed either upstream or downstream of the pump 3. The flowmeter 11 is used for measuring the volumetric flow rate of the drilling fluid in the flowline 50 upstream or downstream of the pump 3. The apparatus 1 also has a fluid densitometer 6 placed either upstream or downstream of the pump 3. The densitometer 6 is used for measuring the density of the drilling fluid in the flowline 50 upstream or downstream of the pump 3. The measured density and volumetric flow rate provide input for calculating the Reynolds number for the flow, e.g. for verifying whether the flow along the flowline is laminar.

The pump 3 can be operated at different pump rates, producing correspondingly, different flow rates for the drilling fluid through the apparatus 1. This can facilitate the measurements of the pressure differentials in the pipe at different flow rates in the fluid.

A valve is typically provided, e.g. on an outlet pipe from the apparatus, to stop flow during gelation periods and/or provide or control a backpressure in the fluid.

The pump 3 is arranged to pump the fluid onward into the large diameter section 12 through a high-shear element in the form of pressure reducing valve 5. The pressure reducing valve 5 produces turbulence, reworks the drilling fluid, reducing the pressure and removing previous fluid structure. The fluid then enters the large diameter section 12 which is configured so that, upon operating the apparatus 1 to pump through fluid, the fluid advances with very low bulk velocity.

In some variants, the apparatus also includes heater/cooler. The heater/cooler is provided upstream of the large diameter section, but typically downstream of the pump. The heater/cooler is operable to heat or cool the drilling fluid. For instance, in use, it can be operated to obtain a particular desired temperature in the fluid. Thus, by operating the heater/cooler to obtain different selected temperatures or temperature ranges, and pumping the fluid through the apparatus at different temperatures, measurement of the differential pressures can take place at different conditions of temperature. An example is described further below.

To facilitate this, the apparatus may further include one or more temperature sensors for obtaining measurements of temperature of the fluid in the apparatus, and in different locations including, especially, along the small diameter section and the medium diameter section.

The first, second, third, and fourth differential pressure sensors 21, 23, 24, 25 can be used to make measurements under various conditions imparted to the fluid due to the pump 3, e.g. at different conditions. They also operate to measure a development of the pressure differential with time.

The apparatus 1 in this example includes a determiner in the form of a computer device 100 for determining the rheological property and/or thixotropy property, using measurements of the differential pressure obtained using the differential pressure sensors.

The computer device 100 is arranged to communicate, e.g. through wired or data connection, with the differential pressure sensors. The computer device 100 is arranged to communicate with the volumetric flow rate and mass density sensors. The computer device 100 is further arranged to communicate with the pump 3 for operating the pump. In the other variants, the computer device 100 also communicates with a cooler/heater, temperature sensor(s).

The computer device 100 is used for controlling the pump 3, e.g. to start and stop the pump. It may also be used to open and close valves for supplying the drilling fluid from the well into the apparatus for measurement.

The computer device 100 has a microprocessor 101 for processing data and executing a computer program for analysing measurements and determining one or more rheological or thixotropy properties of the drilling fluid. The computer device 100 has an In-Out unit 102 for inputting and outputting data to the differential pressure sensors 21, 22, 23, 24, the flowmeter 11, the densitometer 6, and the pump 3. Through the In-Out unit 102, the computer device 100 receives data from the pressure differential sensors, the volumetric flow rate from flow meter, and the fluid mass density from the densitometer. The computer device 100 includes a display 103 for viewing measurement data. Based on the measurement data, the property of the drilling fluid may be determined. The microprocessor 101 is arranged for executing one or more programs for processing and analysing data for producing plots, tables, or models. The computer program may be loaded into memory 104 in the computer device, alternatively remote memory or data storage unit, which is accessed as required, e.g. under instruction from the computer program. The memory 104 is used for storing data, computer programs for performing or facilitating with processing the data for determining the rheological or thixotropy property of the drilling fluid. This may include calculating the Reynolds number.

In use, drilling fluid from the well is supplied into the apparatus through supply pipe 2. The pump 3 is used to pump the drilling fluid through the pressure reducing valve 5 and into the large diameter section 12. The fluid advances downstream through the small diameter section 14, then through the medium diameter section 16. Pressure differentials in the fluid are measured in the small diameter section 14 and the medium diameter section 16. The computer device processes the data from the sensors 21, 22, 23, 24, the flowmeter 11, and the densitometer 6, to determine the rheological or thixotropic property of the drilling fluid. The time dependency of the rheological property is recorded in the data by measuring the pressure differential development over the period of thixotropy after switch on of the pump and/or by comparing the pressure differential in the different upstream and downstream locations from the diameter transitions. The pump rate is changed to another rate, and then the differential pressure measurements can be made again, at the new rate, with the time dependency being obtained by the development in pressure differential after the step up of the pump/flow rate. In the example of FIG. 1, the fluid exiting the medium diameter section then is flowed onward, e.g. to a collection tank and/or onward into the well.

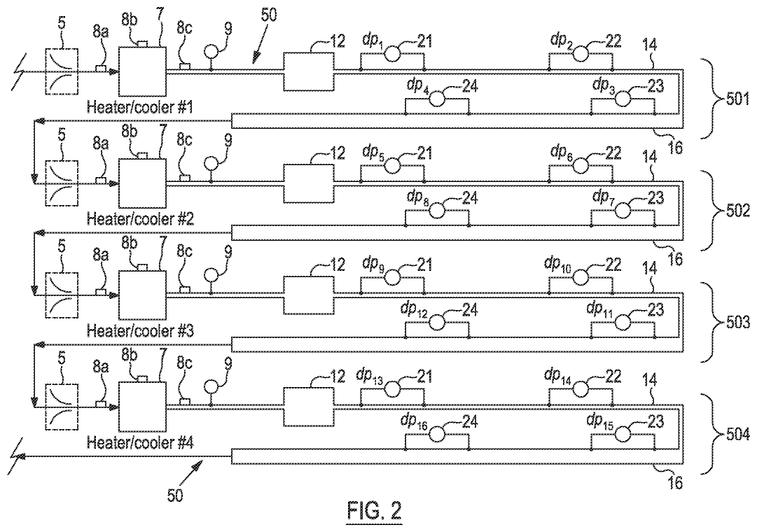

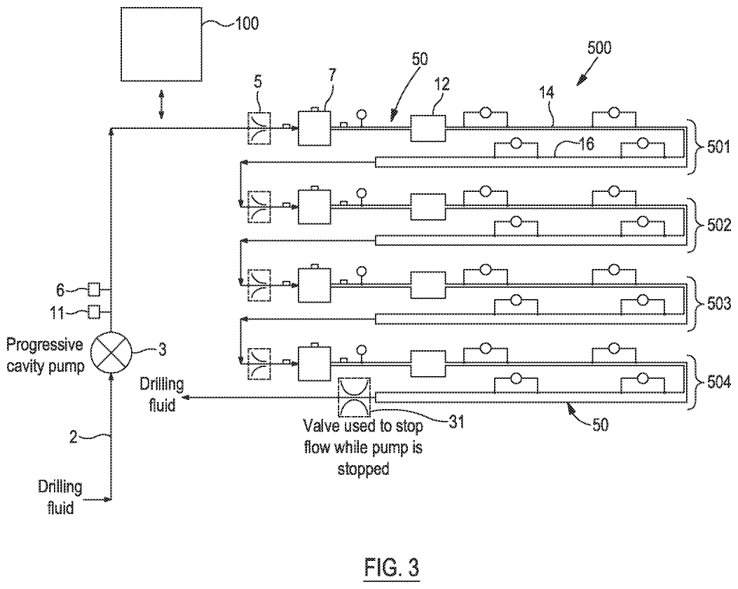

Referring now to FIGS. 2 and 3, another measurement apparatus 500 is generally depicted. The apparatus 500 in this example has several identical pipe rheometer sections 501, 502, 503, 504 connected one after the other. They operate in effect independently to provide independent measurements of the pressure differentials in the drilling fluid which is pumped downstream through a large section, then a small diameter section and then a medium diameter section, the sections in each configured identically and in the same way as in the apparatus 1 described above.

The rheometers 501, 502, 503, 504 in this example, each has a heater/cooler 7 positioned downstream of a pressure reducing valve 5 and upstream of the large diameter section 12. This allows the bulk temperature condition of the flow to be controlled. The different rheometers can then be operated simultaneously to measure pressure differentials using the sensors 21, 22, 23, 24 at different temperatures.

Temperature sensors 8a-8c are provided to obtain measurements of temperature in the fluid passing through the heater/cooler and upstream and downstream of the heater/cooler. The temperature sensors are in communication with the computer device 100 (not shown in FIG. 2). The heaters/coolers 7 are in communication with the computer device 100 (not shown in FIG. 2). The computer device 100 may control the temperature for each of the rheometers 501, 502, 502, 504. The pump (not shown) is also controlled by and in communication with the computer device 100.

An upstream pressure sensor 9 is also provided upstream of the large diameter section 12 in order to measure the bulk pressure in the fluid prior to entry into the measurement sections. A valve 31 is provided for stopping flow e.g. during gelation periods, and/or provide a backpressure in the fluid.

The opening of valve 31 is controlled by the computer 100 to impose a backpressure downstream of the last pipe rheometer. The overall backpressure is measured by the pressure sensor 9 that is also in communication with the computer 100.

In use, drilling fluid is directed along the supply line 2, the pump 3 pumps the fluid into the connected pipe sections of the rheometer 501. The fluid exiting the medium diameter section 16 of the rheometer 501 flows onwards into the next rheometer 502, through the pressure reducing valve 5. The history of the fluid in the earlier rheometer is destroyed through the action of the pressure reducing valve 5. The fluid is heated or cooled to the desired temperature by the heater/cooler 7. Data from the temperature sensors 8a-8c are obtained, and used by computer device 100 to control the heating or cooling. The opening of valve 31 is controlled by the computer device 100 to apply various backpressures downstream of the last rheometer. Data from the pressure sensor 9 are obtained and used by the computer 100 to control the backpressure. The obtaining of the temperature and pressure allows the rheological or thixotropy property of the drilling fluid to be determined using the measurements of differential pressures for particular temperature and pressure conditions, and at different temperature and pressure conditions in the different rheometers 501, 502, 503, 504 of the apparatus. A single pump 3 is used upstream to drive fluid through.

In order to make measurements that record gelation effects, the fluid flow is stopped, by stopping the pump 3, closing the exit-outlet valve 31 and letting the drilling fluid come to rest and form gel. The flow is restarted by starting the pump 3 and controlling the opening of the exit valve 31, and measurements can then be made to determine the rheological property or thixotropy property in the pressure differentials where gelation effects affect the fluid.

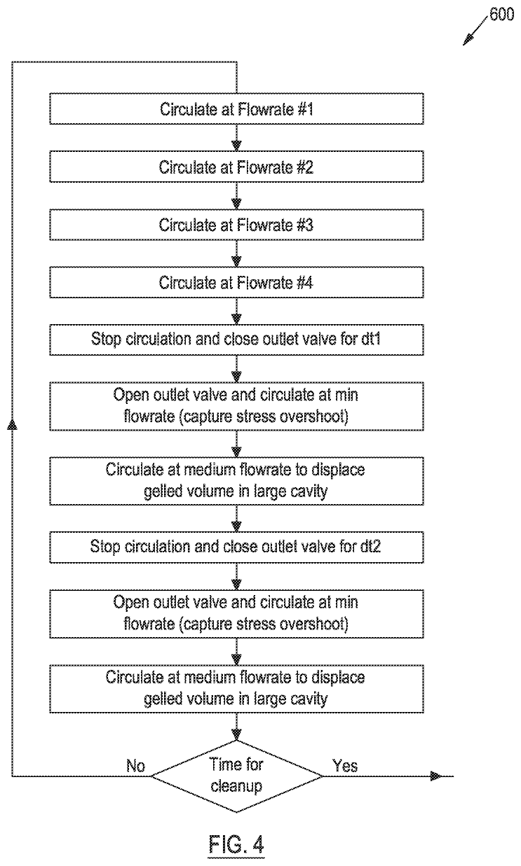

FIG. 4 demonstrates a method 600 of operating the apparatus 500 for obtaining measurements for determining the rheological and thixotropy properties.

### Measurements and Determination of Rheological Properties

The apparatus is configured to allow measurements to be obtained for the determination of rheological properties accounting for thixotropy of the drilling fluid. Measurements are made in various ways to detect the rheological properties of the drilling fluid in the apparatus, in particular, the thixotropy of the drilling fluid, in various ways including:
(i) Measuring the pressure differentials in the drilling fluid upstream and downstream in both the small diameter section 14 and the medium diameter section 16, provides measurements at two spatially separated locations in each of the sections 14, 16. A difference or change in the upstream and downstream pressure differentials between locations, can indicate a difference in rheological property of the drilling fluid at two different distances from the diameter transition D1, D2, and indicate a time dependent rheological behaviour of the fluid;
(ii) The drilling fluid is affected differently in terms of thixotropy if flowing from high to low diameter than if flowing from low to high diameter. Therefore, the measurements of the pressure differentials in the respective small and medium diameter sections 14, 16 reflect the differences in response. Specifically, the measurements of pressure differentials in the small diameter section 14, provide data in response to a transition D1 from high to low diameter, and the measurements of the pressure differentials in the medium diameter section 16, provide data in response to a transition D2 from low to high (medium) diameter.
(iii) The pump 3 may be run at different pump rates, providing different flow rates of the drilling fluid through the sections of the pipe sections of the rheometer. The measurements at each sensor location in (i) and (ii) above are performed over time, i.e. pressure differential data are acquired on an ongoing basis as a time series. Time evolution in the data can then be detected. Upon stepping up the flow rate using the pump 3, the drilling fluid is subject to a new flow and force regime, to which the drilling fluid adapts over time. By obtaining measurements of the pressure differentials at different times relative to the time of stepping up the rate, the evolution over time and rate of adaptation of the fluid can be detected in the measurements that are made. Similarly, the response in pressure differentials after stepping down the pump and flow rate may be measured.
(iv) Gelation effects are also measurable, by letting drilling fluid come to rest and gel inside the apparatus and then, after a period of rest, establishing flow again. Measurements of the pressure differentials in the drilling fluid using the sensors 21, 22, 23, 24 are obtained in time series upon establishing the flow after the rest and gelation of the drilling fluid. The development or evolution in the measurements from one or more of the pressure differential sensors 21, 22, 23, 24 can indicate the time dependence of rheological properties in the course of establishing and initiating the flow of thixotropic drilling fluid from the gel condition. A “stress overshoot” effect may be observed from the pressure differential measurements.
(v) Furthermore, gelation history behaviour is determined using the pressure differential measurements, by obtaining measurements for different durations of rest and thus different degrees of gelling or gel strength. The drilling fluid may be circulated through the rheometer using the pump 3, pumping is stopped and the drilling fluid is left to rest for a certain period of time. Pumping and circulation is then started again, and measurements are made. This is repeated for different periods of rest where gelation takes place. The measurements from the different cycles after different periods of rest can reveal a dependence upon the duration of rest/gelation of the drilling fluid.

The measurements indicated above are performed at for certain bulk temperature and pressure. They can be repeated for different bulk temperatures and pressures which may be controlled by the heater/cooler 7 and the rate of pump 3 and the opening of the exit valve 31 respectively. This is desirable to obtain an indication of the pressure and temperature dependencies of the rheological properties.

In general, it can be noted that the pressure differentials as measured here along the pipeline is indicative of pressure loss gradient along the flow section and of the flow or shear rate of the drilling fluid.

### Predicting Rheological Properties in the Well

While the above measurements allow determination of the rheological properties of the drilling fluid in the measurement environment, e.g. in the topsides equipment of a platform or rig, the pressures and temperatures in the wellbore are considerably different, and it is desirable to determine the properties of the drilling fluid in the wellbore pressure and temperature conditions.

To do this, a model is provided for rheological behaviour of the drilling fluid that takes account of thixotropy, as determined from the measurements mentioned above. The proposed rheological model is a modified non-Newtonian model where an amplification factor is applied to the conventional non-Newtonian relationship to take account of thixotropy. A general form for the non-Newtonian model is:

τ=F(\dot{γ})\dot{γ} (Eq. 1)

where τ is the shear stress, \dot{γ} is the shear rate and FM is a function of the shear rate which is not a constant for non-Newtonian fluids.

An example of a typical non-Newtonian model is the Herschel-Bulkley one expressed as:

τ=τ_{γ}+K\dot{γ}ⁿ (Eq. 2)

where τ_{γ} is the yield stress, K is the consistency index, and n is the flow index. Another example of non-Newtonian model is the Robertson-Stiff model expressed as:

τ=A(\dot{γ}+C)^{B} (Eq. 3)

where A, B and C are the parameters of the model. Yet another example of non-Newtonian model is the Heinz-Casson model expressed as:

τ=(τ_{γ}ⁿ+(K\dot{γ})ⁿ)^{1/n} (Eq. 4)

The impact of thixotropy on the shear stress can be modelled as an amplification factor of the non-Newtonian rheological behaviour as follows:

τ_{\dot{γ},λ}=(1+α(λ−λ_{\dot{γ},∞}))F(\dot{γ})\hat{γ} (Eq. 5)

where λ is a structure parameter that describes the level of structuration inside the fluid and that depends on the shear history, λ_{\dot{γ},∞} is the structure parameter corresponding to the current shear rate \dot{γ} at time ∞, i.e. when thixotropic effects have vanished, α is an amplification function that depends on \dot{γ}ᵢ−\dot{γ}ᵢ₋₁, where \dot{γ}ᵢ is the current shear rate and \dot{γ}ᵢ₋₁ being the previous shear rate. Note that this function tends to the steady state non-Newtonian rheological behaviour when t→∞.

To reconciliate successions of stepping up and down and vice versa, there is considered to be two structure parameters: one that characterizes structuration (λ⁺), i.e. that reflects what happens when the shear rate is stepped down, and one that characterizes de-structuration (λ⁻), i.e. corresponding to stepping up the shear rate. The thixotropy model is then:

$$\{ \begin{matrix}( {τ}_{\dot{γ} , {λ}^{+} , {λ}^{-}} = ( 1 + {α}^{+} ( {λ}^{+} - {λ}_{\dot{γ} , ∞}^{+} ) + {α}^{-} ( {λ}^{-} - {λ}_{\dot{γ} , ∞}^{-} ) ) ( {τ}_{γ , {t}_{g e l}} + K {\dot{γ}}^{n} ) \\ \frac{d {λ}^{+}}{d t} = {{k}_{1}^{+} ( 1 - {λ}^{+} )}^{a} - {k}_{2}^{+} {\dot{γ} ( {λ}^{+} )}^{b} \\ \frac{d {λ}^{-}}{d t} = {{k}_{1}^{-} ( 1 - {λ}^{-} )}^{c} - {k}_{2}^{-} {\dot{γ} ( {λ}^{-} )}^{d}\end{matrix} \qquad ( Eq . 6 )$$

where α⁺ is the amplification function for λ⁺, α⁻ is the amplification function for λ⁻, k₁⁺ and k₂⁺ are the respective parameters of the behavior of λ⁺, k₁⁺ and k₂⁺ are the respective parameters of the behavior of λ⁻, λ_{\dot{γ},∞}⁺ and λ_{\dot{γ},∞}⁻ are respectively the structuration and de-structuration parameters for \dot{γ} when t→∞, a, b, c and d are rational exponents that may be fitted to the type of fluid being measured by the apparatus.

The amplification function α⁺ and α⁻ may be approximated to polynomial functions of \dot{γ}ᵢ−\dot{γ}ᵢ₋₁. For instance, quadratic approximations are expressed as:

$$\{ {\begin{matrix}{α}^{+} = {α}_{0}^{+} + {α}_{1}^{+} ( {\dot{γ}}_{i} - {\dot{γ}}_{i - 1} ) + {α}_{2}^{+} ( {\dot{γ}}_{i} - {\dot{γ}}_{i - 1} ) \\ {α}^{-} = {α}_{0}^{-} + {α}_{1}^{-} ( {\dot{γ}}_{i} - {\dot{γ}}_{i - 1} ) + {α}_{2}^{-} {( {\dot{γ}}_{i} - {\dot{γ}}_{i - 1} )}^{2}\end{matrix}}^{2} \qquad ( Eq . 7 )$$

with α₀⁺, α₁⁺, α₂⁺, α₀⁻, α₁⁻ and α₂⁻ being parameters that can be calibrated.

Thus, the complete time-dependent rheological behaviour model has 18 parameters: τ_{gel}(t₁), τ_{gel}(t₂), M₁, α₁, α₂, τ_{γ}, K, n, k₁⁺, k₂⁺, k₁⁻, k₂⁻, α₀⁺, α₁⁺, α₂⁺, α₀⁻, α₁⁻, α₂⁻.

In this example, to calibrate the 13 parameters (τ_{γ}, K, n, k₁⁺, k₂⁺, k₁⁻, k₂⁻, α₀⁺, α₁⁺, α₂⁺, α₀⁻, α₁⁻, α₂⁻) of the model that characterizes the thixotropic behavior of the fluid when gelation effects have dissipated, it is necessary to use at least four different flow rates in the rheometer (four flow rates x four differential pressures (from the sensors 21, 22, 23, 24)>13). These flow rates are chosen to give shear rates at the wall along the two pipe sections that are preferably in the region of 1 to 300 s⁻¹. Calculation of the parameters may be performed by the computer device or other determiner.

The measurements from the pipe rheometer(s) described above allow the coefficients the model to be determined to provide a calibrated model fitting the measurement data. The model can be in the form of “flow curve” of shear stress against shear rates which provides an indication beyond the low shear rate region of the measurements, and into a region relevant for typical wellbore conditions. The flow curve can also be determined as a function of temperature and pressure. Based on this curve, the rheological property of the drilling fluid in well conditions can be obtained.

### Advantages

The pipe rheometer provides for obtaining a comprehensive set of measurements for determining the rheological properties and thixotropy of a drilling fluid. Moreover, it is suitable to cope with drilling fluids directly from the well with lost circulation materials and solids content. By taking into account thixotropy in the various ways measurements are performed, a more accurate characterisation of the drilling fluid in wellbore environment may be obtained, because the models for extrapolating the rheological behaviour toward other wellbore conditions, have a foundation in correct fitting to the measurement data at low shear rates where thixotropy effects are prevalent and have greatest percentage impact on values, where previously such effects e.g. by use of simple Herschel-Bulkley rheological behaviour without accounting for thixotropy may not be suitable or accurate. It can be conveniently used to obtain measurement of fluids in a continuous manner as part of larger fluid flow or circulation system by directing flow into and out of the pipe rheometer. Thus, “live” measurements may be obtained for the system.

Although various examples and aspects above have been specified with reference to drilling fluid, the techniques could be applied correspondingly to other fluids which may have the same or similar characteristics or complexities of behaviour, such as paint, yoghurt/soup, ketchup, blood, cement slurry, etc. In alternative examples therefore, the fluid which is pumped through the rheometer to determine the rheological or thixotropy property, is paint, yoghurt/soup, ketchup, blood, or cement slurry, or other fluid, instead of drilling fluid.

1.-34. (canceled)

35. A method of determining at least one rheological property, for example a thixotropy property, of a drilling fluid, the method comprising the steps of:
providing measurement apparatus comprising a flowline comprising a first diameter section and a second diameter section;
pumping the drilling fluid along the flowline, subjecting the drilling fluid to different shear conditions;
measuring at least one upstream differential pressure in the drilling fluid upstream in the second diameter section, using at least one upstream sensor, and measuring at least one downstream differential pressure in the drilling fluid downstream in the second diameter section, using at least one downstream sensor, whereby thixotropic dependency upon shear history of the drilling fluid may be obtained in the measured differential pressures; and
using the upstream and downstream differential pressures with the shear history dependency therein to determine the rheological property and/or the thixotropy property.

36. The method as claimed in claim 35, wherein the drilling fluid is pumped through at least one diameter transition in the flowline.

37. The method as claimed in claim 35, wherein the first diameter section has a different diameter from the second diameter section.

38. The method as claimed in claim 37, wherein the first diameter section is a large diameter section and the second diameter section is a small diameter section, and the method further comprises using the differential pressures from the second diameter section to determine thixotropic behaviour of the drilling fluid for increasing shear rates.

39. The method as claimed in claim 38, wherein the flowline includes a third diameter section which is a different diameter section having a diameter larger than the second diameter section, and the method further comprises:
communicating the drilling fluid through the first diameter section, thereafter the second diameter section, thereafter the third diameter section;
measuring at least one upstream differential pressure in the drilling fluid upstream in the third diameter section using at least one upstream sensor;
measuring at least one downstream differential pressure in the drilling fluid downstream in the third diameter section using at least one downstream sensor; and
using the upstream and downstream differential pressures from the third diameter section to determine the rheological property and/or thixotropic behaviour for decreasing shear rates.

40. The method as claimed in claim 35, which further comprises:
using at least one pump to pump the drilling fluid along the flowline;
changing the pump rate of the pump from a first pump rate to a second pump rate;
obtaining measurements of the differential pressures at the first pump rate; and
after changing the pump rate, obtaining measurements of the differential pressures at the second pump rate.

41. The method as claimed in claim 35, which further comprises:
stepping up the flow rate along the flowline, using at least one pump;
obtaining a temporal development in at least some of the measured differential pressures in response to the change of flow rate;
using the temporal development to determine the thixotropic property of the drilling fluid.

42. The method as claimed in claim 35, which further comprises:
heating or cooling the fluid to change temperature conditions of the drilling fluid in the flowline; and
performing the measurements of the differential pressures before and after changing the temperature conditions.

43. The method as claimed in claim 35, which further comprises operating a choke to produce a back pressure in the drilling fluid in the flowline.

44. The method as claimed in claim 35, which further comprises:
letting the fluid rest to produce gelation of the drilling fluid in a section of the flowline; and
after gelation:
starting the pumping of the drilling fluid;
directing the drilling fluid with the gelation history into the second diameter section; and
obtaining data comprising a temporal development, the data obtained from either or both the first and second pressure differential sensors; and
using the data to obtain the thixotropy property of the fluid with the gelation history.

45. The method as claimed in claim 35, which further comprises:
providing a rheological model, the model preferably having a thixotropy component;
using the rheological model and the measurements obtained from the measurement apparatus to determining the rheological property of the drilling fluid in conditions of the work environment.

46. The method as claimed in claim 45, wherein the model comprises a thixotropic non-Newtonian rheological model.

47. An apparatus for performing the method to determine at least one rheological property, for example a thixotropy property, of a drilling fluid, the apparatus comprising:
a flowline comprising a first diameter section and a second diameter section;
pressure sensors for measuring at least one upstream pressure differential and at least one downstream pressure differential in the second diameter section of the conduit; and
determiner means for determining the rheological property, or the thixotropy property, of the drilling fluid based on the measured upstream and downstream pressure differentials.

48. The apparatus as claimed in claim 47, further comprising at least one pump for pumping the drilling fluid through the flowline, the pump operable to have at least one pump rate for controlling pressure conditions and flow of the drilling fluid through the flowline.

49. The apparatus as claimed in claim 47, further comprising at least one heater/cooler for changing temperature conditions of the drilling fluid in the flowline.

50. The apparatus as claimed in claim 47, further comprising at least one temperature sensor for obtaining at least one measurement of temperature of the drilling fluid in the flowline.

51. The apparatus as claimed in claim 47, further comprising an element, e.g. valve, for producing turbulence, shearing and/or reducing pressure in the drilling fluid upstream of first and second diameter sections of the flowline for producing low bulk velocity drilling fluid.

52. The apparatus as claimed in claim 47, further comprising means for determining at least one gelation property of the drilling fluid at different bulk temperatures and pressures of the drilling fluid in the flowline.

53. The apparatus as claimed in claim 47, wherein the flowline comprises first and second diameter sections, wherein the first diameter section comprises a large diameter section with a diameter greater than the second diameter section, and optionally greater than a third diameter section, for obtaining sheared fluid in the large diameter section at a low bulk velocity.

54. The apparatus as claimed in claim 47, further comprising means for determining the density of the fluid in the conduit.

55. A wellbore drilling system wherein the apparatus is connected to a drilling fluid circulation system for circulating drilling fluid into and out of a wellbore for drilling operations, wherein the flowline of the apparatus is arranged in fluid communication with the wellbore for supplying or receiving drilling fluid from the borehole into the flowline of the measurement apparatus for measurement and/or communicating the measured drilling fluid from the apparatus onward for use in the wellbore.

56. A method of determining at least one gelation property of a drilling fluid, the method comprising:
providing measurement apparatus comprising a flowline;
entering the drilling fluid into the flowline;
letting the drilling fluid rest in the flowline, producing thereby gelation of the drilling fluid;
pumping the drilling fluid through and along the flowline after a period of rest and gelation; and
obtaining measurements of at least one property of the drilling fluid in the flowline to determine the gelation property;
the flowline comprises first and second diameter sections, and the method further comprises:
measuring at least one upstream differential pressure in the drilling fluid upstream in at least the second diameter section using at least one upstream sensor;
measuring at least one downstream differential pressure in the drilling fluid downstream in at least the second diameter section using at least one downstream sensor; and
using the measurements of the upstream and downstream differential pressures to determine the gelation property.

57. The method as claimed in claim 56, which further comprises:
starting the pumping of the drilling fluid after a period of rest and gelation of the drilling fluid; and
using a time evolution of measured pressure differentials after the pumping is started, to determine the gelation property.

58. The method as claimed in claim 56, which further comprises stopping the pumping to place the drilling fluid at rest in the apparatus for producing gelation.

59. The method as claimed in claim 56, which further comprises, repeatedly:
stopping pumping to place the drilling fluid at rest in the apparatus for producing gelation;
starting the pumping of the drilling period after a period of rest of the drilling fluid; and
measuring a time evolution of the pressure differentials after the pumping is started, to determine the gelation property.

60. The method as claimed in claim 59, wherein the repeated steps further include measuring the duration of rest.

61. The method as claimed in claim 59, which further comprises measuring the time evolution of the pressure differentials for different durations of rest.

62. The method as claimed in claim 58, which further comprises obtaining the measurements at different conditions of pressure and temperature in the apparatus.

63. The method as claimed in claim 58, which further includes operating at least one pump to control either or both of:
pressure conditions; and
flow and rest periods of the drilling fluid.

64. The method as claimed in any of claim 58, which further comprises producing turbulence prior to entering a large diameter section by which the sheared fluid is subject to very low bulk velocity, for thereafter entering the second diameter section and then passing through into a third diameter section.
